(19) United States
(12) Patent Application Publication
(10) Pub. No.: US 2021/0231668 A1
(43) Pub. Date: Jul. 29, 2021

(54) KIT AND METHOD FOR DETERMINING PROSTATE CANCER MALIGNANCY

(71) Applicant: J-Pharma Co., Ltd., Kanagawa (JP)

(72) Inventors: Nobuyuki Yanagisawa, Kanagawa (JP); Isao Okayasu, Tokyo (JP)

(73) Assignee: J-Pharma Co., Ltd., Kanagawa (JP)

(21) Appl. No.: 17/169,033

(22) Filed: Feb. 5, 2021

Related U.S. Application Data
(62) Division of application No. 16/328,214, filed on Feb. 25, 2019.
(60) Provisional application No. 62/131,679, filed on Mar. 11, 2015.

(51) Int. Cl. G01N 33/574; C07K 16/28; G01N 33/53

(52) U.S. Cl. CPC G01N 33/57434; C07K 16/28; G01N 2333/705; G01N 2800/56; G01N 33/53

(57) ABSTRACT

The present invention relates to kits and methods for determining (diagnosing) prostate cancer malignancy and to predict patient prognoses. Our findings suggested that elevated LAT1 expression in PC is a novel biomarker for high-grade malignancy. Independently of GS, aberrant LAT1 overexpression might be used to screen for aggressive phenotypes of PC that should be treated medically. Prostate biopsies are usually small samples, limiting the evaluation of the tumor area. Thus, LAT1 intensity in prostate biopsy samples may be more a reliable prognostic marker of LP. Especially, we propose LAT1 evaluation against PC with low-risk patients in order to screen who can receive active surveillance. Several LAT1 inhibitors have been found to suppress cancer cell proliferation, so inhibition of LAT1 may be a potential therapeutic strategy for PC and other human cancers.

### TECHNICAL FIELD

The present invention relates to kits and methods for determining (diagnosing) prostate cancer malignancy and predicting prognoses in patients.

### BACKGROUND ART

Prostatic cancer (PC) is the most common nonskin cancer affecting men in the United States,[1] but its natural history is variable and frequently indolent. Histologically, Gleason score (GS) is one of the most powerful predictors of PC patient prognosis.[2; 3; 4] Moreover, GS is currently the most widely accepted histologic grading method and one of the most important predictors provided by prostate needle biopsies.[5; 6] Other pathologic characteristics in prostate biopsies used to predict prostate-specific antigen (PSA)-free recurrence include number of biopsy cores containing cancer,[7] length or percentage of lesion in each biopsy core containing cancer,[8; 9] presence of perineural invasion[10] and amount of reactive stroma.[11] Prostate biopsies can evaluate PC before therapeutic interventions such as radical prostatectomy, radiation therapy, or neoadjuvant/adjuvant therapy. However, it is often difficult to evaluate biomarkers correctly in prostatic biopsy specimens, because these samples are small and provide limited information.[12; 13] Additional biomarkers in biopsy samples may improve the predictive ability to manage patients.

Active surveillance (AS) may be suitable for patients who later undergo a curative approach.[14] These patients, with very low or low risk PC are initially not treated but are followed-up periodically. If AS shows progression or threat of progression, these patients undergo treatment with curative intent. AS is used to reduce overtreatment of patients with clinically confined very low and low-risk PC. In contrast, watchful waiting (WW) is used to monitor patients with locally advanced PC for whom local therapy is not mandatory; WW is considered an alternative to androgen-deprivation therapy, with equivalent oncologic efficacy.[14]

### SUMMARY OF INVENTION

### Technical Problem

However, the balance between intervention and overtreatment may be difficult to determine and may depend on patient age, comorbidities, performance status, life expectancy and clinicopathological factors including biopsy GS. Moreover, most AS protocols use PSA kinetics as a trigger to initiate aggressive treatment. However, PSA kinetics alone, including PSA-doubling time (PSADT) and PSA velocity, are not reliable triggers for treatment intervention.[15] Identification of new biomarkers may better predict the lethality of PC. Accordingly, an object of the present invention is, independently of GS, to provide a reliable prognostic marker of local progression (LP), to provide means capable of determining prostate cancer malignancy more accurately and easily, and also to provide evaluation against PC with low-risk patients in order to screen who can receive active surveillance.

### Solution to Problem

The present invention (1) is a kit, comprising an anti-human LAT1 monoclonal antibody, used to determine prostate cancer malignancy via immunohistochemical staining.

The present invention (2) is the kit used to determine prostate cancer malignancy according to the present invention (1), wherein the monoclonal antibody recognizes human LAT1 amino acid residues specifically at positions 1 to 52 from the N-terminus.

The present invention (3) is the kit used to determine prostate cancer malignancy according to the present invention (1), which is used for a patient associated with low-risk in prognosis.

The present invention (4) is a method for determining prostate cancer malignancy by means of immunohistochemical staining, which comprises a step of applying an anti-human LAT1 monoclonal antibody to a specimen tissue.

The present invention (5) is the method for determining prostate cancer malignancy according to present invention (4), wherein the monoclonal antibody recognizes human LAT1 amino acid residues specifically at positions 1 to 52 from the N-terminus.

The present invention (6) is the method for determining prostate cancer malignancy according to present invention (4), which is used for a patient associated with low-risk in prognosis.

The present invention (7) is a method to clinically differentiate prostate cancer severity via application of LAT1 molecular target therapeutic agent(s), which comprises a step of determining malignancy of prostate cancer according to the method as claimed in the present invention (4), (5) or (6) and a step of determining whether a therapeutic agent for prostate cancer is to be administered or not, based on the diagnosis result.

### Advantageous Effects of Invention

According to the present invention, it is possible to provide, independently of GS, a reliable prognostic marker of LP, to provide means capable of determining prostate cancer malignancy more accurately and easily, and also to provide evaluation against PC with low-risk patients in order to screen who can receive active surveillance.

### BRIEF DESCRIPTION OF DRAWINGS

FIG. 1A-FIG. 1D L-type amino acid transporter (LAT) 1 expression in prostate carcinoma (PC) cells analyzed by immunohistochemistry. The immunointensity of the carcinoma cell membrane was categorized as A, 0, no staining; B, 1, weak or patchily positive staining; C, 2, moderate cell membrane staining; and D, 3, intense complete membrane staining. Activated lymphocytes also showed LAT1 expression. Slides were counterstained with methyl green solution. Original magnification, ×400 (A-D).

FIG. 2A-FIG. 2E Comparison of LAT1 scores and intensities in prostate cancer patients with local progression (LP) and stable disease (SD). LAT1 expression was significantly higher in LP than in SD patients. A, LAT1 scores; B, LAT1 intensities; C, Gleason score (GS) 7 lesions; D, LAT1 expression in prostate cancer patients divided by D'Amico risk categories (low-, intermediate- and high-risk groups). Within each category, LAT1 expression was greater in LP than in SD groups. E, Comparison of LAT1, LAT2, CD98 expressions and Ki-67 labeling index (LI) in GS-low (GS<7) patients. Only LAT1 expression was significantly higher in LP than in SD patients. *p<0.0001, #p<0.01, ¶p<0.05.

### DESCRIPTION OF EMBODIMENTS

In order to solve the above problem, the inventors first focused attention on amino acid transporter LAT1 which is expressed specifically to cancer-derived culture cells and fetal livers. L-type amino acid transporters (LATs) are responsible for the transport of large neutral amino acids. Most of these transporters consist of two subunits, a light chain, including LAT1 (SLC7A5) and LAT2 (SLC7A8), and a heavy chain (CD98/4F2hc), located in the cell membrane.[16; 17] LAT2 is widely expressed in normal cells, such as small intestine epithelial cells and proximal tubules of the kidney, suggesting that it plays an important role in active transepithelial transport of amino acids.[16] In contrast, LAT1 is expressed in many carcinoma cells, including prostatic, gastric, pulmonary and pancreatic carcinomas.[18; 19; 20; 21] Furthermore, some fetal cells express LAT1, suggesting that LAT1 may be an oncofetal protein.[22] We recently reported that high LAT1 expression was predictive of poorer prognosis in patients with pancreatic ductal adenocarcinomas and bile duct adenocarcinomas, independent of cellular proliferation activity according to Ki-67 labeling index (LI).[21; 23] These findings strongly suggested that high levels of LAT1 expression are associated with aggressive phenotypes of malignant tumors.

Several clinical trials targeting LAT1 have been started at different medical institutions. A LAT1 inhibitor, JPH203, is going to apply to human malignancies.[24; 25] In addition, NMK36, novel positron emission tomography (PET) radiotracer containing a synthetic amino acid analogue anti-1-amino-3-¹⁸F-fluorocyclobutane-1-carboxylic acid (FACBC), is well taken up by tumor cells through LAT1. The results of this phase IIa clinical trial indicated the potential of anti-¹⁸F-FACBC PET to delineate primary PC lesions and metastatic lesions, and currently ongoing this phase IIb trial (registered as JapicCTI-121807).[26; 27] Therefore LAT1 could become an important molecular target of effective agents in therapy as well as diagnosis against human malignancies.

We therefore assayed LAT1 expression in PC biopsy samples of patients undergoing EM to determine whether altered LAT1 expression is related to the malignant behavior of PCs.

The present invention (1) is a kit for determining malignancy of prostate cancer by means of immunohistochemical staining, which comprises an anti-human LAT1 monoclonal antibody. Herein, such anti-human LAT1 monoclonal antibodies are not particularly limited as long as they can specifically recognize LAT1; examples of which may include antibodies which specifically recognize amino acid residues at positions 1 to 52 from the N-terminus of the intracellular region of LAT1 (Met Ala Gly Ala Gly Pro Lys Arg Arg Ala Leu Ala Ala Pro Ala Ala Glu Glu Lys Glu Glu Ala Arg Glu Lys Met Leu Ala Ala Lys Ser Ala Asp Gly Ser Ala Pro Ala Gly Glu Gly Glu Gly Val Thr Leu Gln Arg Asn Ile Thr Lue) (for example, human LAT1 mouse monoclonal antibodies). The amino acid sequence and the base sequence of human LAT1 are described in Japanese Unexamined Patent Publication No. 2000-157286. In addition, in the context of the term “malignancy” as used herein, cancer has severe malignancy when a patient dies due to prostate cancer and mildly malignant when a patient, even if diagnosed with cancer, does not die directly due to prostate cancer.

Herein, anti-human LAT1 monoclonal antibodies are not particularly limited as long as they take LAT1 as antigens and bind to such antigens. Therefore, mouse antibodies, rat antibodies, rabbit antibodies, sheep antibodies and the like may appropriately be used.

Also, hybridomas producing monoclonal antibodies can be produced, basically using known techniques as follows. Specifically, monoclonal antibodies may be produced by using desired antigens and/or cells expressing such desired antigens as sensitized antigens, immunizing them according to conventional immunization methods, fusing the obtained immunocytes with known parent cells by means of conventional cell fusion methods and screening monoclonal antibody-producing cells (hybridomas) by means of conventional screening methods. Production of hybridomas may be carried out, for example, according to the method of Milstein et al. (Kohler, G. and Milstein, C., Methods Enzymol. (1981) 73: 3-46), and the like. In producing anti-human LAT1 monoclonal antibodies, LAT1 or fragments of the protein may be used as antigens; thus, LAT1 or cells expressing fragments of the protein may also be used as antigens. LAT1 or fragments of the protein may be obtained, for example, according to the method described in Molecular Cloning: A Laboratory Manual, 2nd. Ed., Vols. 1-3, Sambrook, J. et al, Cold Spring Harbor Laboratory Press, New York, 1989. Also LAT1 or cells expressing fragments of the protein may be obtained according to the method described in Molecular Cloning: A Laboratory Manual, 2nd. Ed., Vols. 1-3, Sambrook, J. et al, Cold Spring Harbor Laboratory Press, New York, 1989.

The kit may also include additional components, such as:

(1) an antibody labeled with peroxidase for anti-human LAT1 monoclonal antibody,

(2) a peroxide which inhibits endogenous peroxidase,

(3) a redox dye which develops a color via oxidization,

(4) an activator reagent for facilitating bonding between an antigen protein (LAT1) and an antibody,

(5) a blocking reagent which inhibits nonspecific bonding between proteins other than LAT1 in tissues and an antibody, and

(6) a cleaning agent for removing reagents attached to specimens at each step.

Regarding redox dye (3; above), while there are a number of signals whose intensities may be measured (for example, fluorescence), color changes in the visible light region are required. Reasons for this are not clear, but in case of other signals, use of the anti-human LAT1 monoclonal antibody according to the present invention does not provide sufficient distinction between benign versus malignant prostate cancer. On the other hand, using the anti-human LAT1 monoclonal antibody according to the present invention in combination with a reagent which enables observation of color changes within visible light regions (i.e. immunohistochemical staining), distinction between benign and malignant prostate cancer may clearly be defined.

The present invention (4) is a method to determine prostate cancer malignancy by means of immunohistochemical staining, which comprises a step of applying an anti-human LAT1 monoclonal antibody to a specimen tissue.

Herein, the method may additionally include any or all of the following steps:

a step of applying a peroxide to the specimen tissue,

a step of immersing the specimen tissue in an activator reagent and applying microwave treatment,

a step of applying a blocking reagent to the specimen tissue,

a step of applying a labeled antibody for the anti-human LAT1 monoclonal antibody,

a step of applying a redox dye which develops a color via oxidization, and

optionally, a step of applying a primary antibody negative control to the specimen tissue.

The present invention (7) also is a method of differentiating prostate cancer cases via application of LAT1 molecular target therapeutic agent(s), which comprises a step of determining prostate cancer malignancy according to the method of the invention (2) and a step of determining whether or not a therapeutic agent for prostate cancer be administered based upon the diagnosis result.

### EXAMPLES

### Materials and Methods

### Production Example 1 Example of Primary Antibody Production

The primary antibody (2.0 μg protein/ml) contains anti-human L-type amino acid transporter 1 (hLAT1) mouse monoclonal antibody. The antibody was made by using the proteins at positions 1 to 52 of hLAT1 synthesized by hLAT1 cloning vectors according to the in vitro translation method as antigens to immunize BALB/c mice and then fusing their spleen cells with mouse myeloma cells to obtain hybridomas, which were intraperitoneally inoculated to mice to obtain ascites fluid, which was purified by ammonium sulfate fractionation and Protein G coupling column chromatography and dissolved in 10 mM PBS (pH 7.4) containing 1% bovine serum albumin. The LAT1 amino acid sequence and the base sequence coding the protein are described in Japanese Unexamined Patent Publication No. 2000-157286.

### Production Example 2 Example of Determination Kit Composition

The determination kit according to the present invention is composed of the following six reagents.

Blocking reagent; prepared by diluting normal swine serum to 2%.

Primary antibody; prepared by diluting an anti-LAT1 mouse monoclonal antibody (Production Example 1) to 2 μg/mL with a buffer (1% BSA, 0.25% casein sodium, 15 mM sodium azide, 0.1% Tween 20).

Polymer reagent; Nichirei Histofine Simple Stain MAX-PO(M)™. This reagent contains 4 μg/mL of peroxidase-labeled anti-mouse IgG goat polyclonal antibody (Fab′).

Primary antibody negative control; Mouse IgG (Vector Laboratories) gets dissolved in the buffer described above to 2 μg/mL.

Substrate buffer; Tris[hydroxyl methyl]amino methane and tris[hydroxyl methyl]amino methane are diluted with distilled water; and,

Coloring substrate; DAB (3-3′Diaminobendine tetrahydrochloride) dissolved in a buffer (substrate buffer described above) to 0.2 mg/mL.

The determination kit according this Production Example may further contain the following reagents used for staining.

Endogenous peroxidase blocking reagent: 1% H₂O₂/methanol

Aqueous hydrogen peroxide is diluted with methanol to 1%.

Activator reagent: 0.01 M citrate buffer (pH 6.0)

Citric acid monohydrate (0.36 g) and trisodium citrate dihydrate (2.44 g) are dissolved in distilled water to 1 L.

Cleaning solution: PBS

Disodium hydrogen phosphate 12-water (2.90 g), sodium dihydrogen phosphate dihydrate (0.296 g) and sodium chloride (8.5 g) are dissolved in distilled water to 1 L.

To sum up the above, the reagents composing the diagnosis kit according to this Production Example (six essential reagents) are shown in Table 1 below.

TABLE 1
Example of reagents composing the diagnosis kit
according to this Production Example

| | Kit for | | Kit for automatic |
|---|---|---|---|
| | manual | | immunostaining |
| Reagents | method | | device |
| Blocking reagent | 7.0 | mL | 2 × 11 mL |
| (2% swine serum) | | | |
| Primary antibody | 3.5 | mL | 1 × 15 mL |
| (containing 2 μg/mL of | | | |
| anti-hLAT1 antibody) | | | |
| Polymer reagent (Simple | 7.0 | mL | 2 × 11 mL |
| Stain MAX PO (M) ) | | | |
| Primary antibody negative | 3.5 | mL | 1 × 15 mL |
| control (mouse IgG) | | | |
| Substrate buffer | 10 | mL | 15 × 11 mL |
| (Tris buffer) | | | |
| coloring substrate (0.2 | 0.5 | mL | 2 × 2 mL |
| mg/mL DAB solution) | | | |

Method for Operation and Method for Determination

1. Method for Operation

Procedures for operation are summarized in Table 2.

TABLE 2
Immunohistochemical detection system

| Steps | | Reagents | Operations |
|---|---|---|---|
| 1 | Endogenous | 1% H₂O₂ | Treat for 30 min at room |
| | peroxidase | | temperature under moist |
| | activation | | conditions |
| 2 | Antigen | Antigen | Microwave for 5 min and |
| | activation | activator | then treat for 20 min at |
| | | solution | room temperature |
| 3 | Blocking | Blocking reagent | Treat for 30 min at room |
| | | | temperature under moist |
| | | | conditions |
| 4 | Primary | Refer to Matters | Treat for 1 hr at room |
| | antibody | to be Considered | temperature under moist |
| | treatment | | conditions |
| 5 | Secondary | Nichirei | Treat for 30 min at room |
| | antibody | Histofine Simple | temperature under moist |
| | treatment | Stain MAX PO | conditions |
| | | (M) | |
| 6 | DAB coloring | Coloring | Treat for 15 min at room |
| | | substrate | temperature |
| | | solution | |
| 7 | counter- | Hematoxylin | Treat for 1 min at room |
| | staining | | temperature and then |
| | | | wash with water |

1-1. Method for Manual Operation

After deparaffinization, a specimen tissue slide is immersed in an endogenous peroxidase blocking reagent in a staining vat, treated for 30 minutes at room temperature and then washed with water. Excess moisture is removed from the specimen and the specimen is immersed in an activator reagent and then microwaved for five minutes. After the treatment, the specimen is sufficiently cooled down to room temperature and then washed with water and further with a cleaning solution. Excess moisture is removed from the specimen and a sufficient amount of blocking reagent to be uniformly distributed is added dropwise to the tissue section and allowed to react for 30 minutes at room temperature in a moist chamber. Excess moisture is removed from the specimen and a sufficient amount of primary antibody is added dropwise and allowed to react for one hour at room temperature in a moist chamber, followed by washing with a cleaning solution (three times each for five minutes). To a specimen tissue slide for negative control, a sufficient amount of primary antibody negative control is added dropwise, instead of the primary antibody, for similar treatment. Excess moisture is removed from the specimen and a sufficient amount of polymer reagent is added dropwise and allowed to react for 30 minutes at room temperature in a moist chamber, followed by washing with a cleaning solution (three times each for five minutes). Excess moisture is removed from the specimen and a predetermined amount of substrate solution is added dropwise to or immersed in the specimen and allowed to react for 15 minutes at room temperature in a moist chamber or staining pot, followed by washing with a cleaning solution. The specimen is stained with a counterstaining liquor (for example, Mayer's hematoxylin liquor) followed by washing with water. After dehydration with an alcohol series and substitution with xylene, the specimen is mounted for use in microscopy.

1-2. Method for Operation with Automatic Immunostaining Device

A specimen tissue slide, blocking reagent, primary antibody, primary antibody negative control, polymer reagent, substrate solution, distilled water, cleaning solution and counterstaining liquor are placed at predetermined locations and the reagents are allowed to react for a predetermined period of time at room temperature under moist conditions. Water of the specimen is substituted with an alcohol and then with xylene and the specimen is then mounted for use in microscopy.

### <Patients, Follow-Up and Tissue Samples>

This study involved 109 men diagnosed with prostatic adenocarcinoma between 1991 and 2006 and undergoing EM at Kitasato University Hospital. Their diagnoses were established from histologic examination of prostate biopsies or transurethral resections (TUR), and the histology was reviewed and re-graded according to the Gleason system by one pathologist (N.Y.). Patients were staged according to the 2009 revised TNM classification.[28] Other details about our study patients are given in Table 3.

TABLE 3
Patient charactaristics

| | Stable | Local | |
|---|---|---|---|
| | cases | progression | |
| | (n = 65) | cases (n = 44) | p |
| Age | 73.9 ± 7.6 | 73.9 ± 5.2 | 0.6831 |
| | (53-87) | (60-83) | |
| Gleason score (GS) | 7.0 ± 0.8 | 7.3 ± 0.8 | 0.0744 |
| GS6/7/8/9 | 19/29/14/3 | 6/22/13/3 | |
| Initial PSA (ng/ml) | 10.5 ± 16.6 | 22.4 ± 35.7 | 0.0015* |
| | (0.4-124) | (0.7-203) | |
| Clinical TNM stage | 51/13/1/0 | 22/16/6/0 | |
| I/II/III/IV | | | |
| D'Amico risk | 15/30/20 | 4/16/24 | |
| categories | | | |
| Low/Int/High | | | |

Abbreviations: GS, Gleason score;
Int, intermediate;
PSA, prostate-specific antigen.
*p value indicates a significant difference.

Patients not receiving medical treatment were followed for at least 12 months (average 80 months; range, 13-215 months) after their first biopsy, with PSA measured at least 3 times. Serum PSA levels were monitored every 3 months. Local progression (LP) was defined as an increase in clinical T stage by digital rectal examination and/or by radiological examinations, as reported previously.[29] All patients underwent chest X-rays, CT scan or MRI of the abdominal/pelvic cavity and bone scintigraphy at least once per year to rule out the presence of metastases.

Tissue samples were those obtained by prostatic biopsy or TUR at the initial diagnosis of adenocarcinoma. All of these specimens had been fixed in 10% buffered formalin and embedded in paraffin. One or two cancer-containing biopsy cores or TUR chips from each patient were selected and used for hematoxylin-eosin staining and immunohistochemical analyses. A total of 172 PC lesions from the 109 PC patients were examined.

### <Immunohistochemistry>

Tissue sections 4 μm thick were stained immunohistochemically as described.[20; 23] Briefly, endogenous peroxidase was blocked with 1% hydrogen peroxide in methanol for 30 minutes. Following antigen retrieval, the sections were incubated overnight at 4° C. with primary antibodies, including mouse monoclonal anti-LAT1 (2 μg/ml, J-Pharma Co., Ltd., Kanagawa, Japan), rabbit polyclonal anti-LAT2 (2 μg/ml, Trans Genic Inc., Kumamoto, Japan), mouse monoclonal anti-CD98 (clone H-300, 1:200, Santa Cruz Biotechnology Inc., Dallas, Tex.) and mouse monoclonal anti-Ki-67 (1:100, Dako, Glostrup, Denmark). The antigenic specificities of the anti-LAT1 and anti-LAT2 antibodies had been previously confirmed.[20; 30] After incubation with peroxidase-labeled polymer (ChemMate EnVision kit, Dako) for 30 minutes, the samples were incubated with the chromogen 3,3′-diaminobenzidine (DAB). Nuclei were counter-stained with 0.3% methyl green.

### <Evaluation of Immunohistochemical Staining>

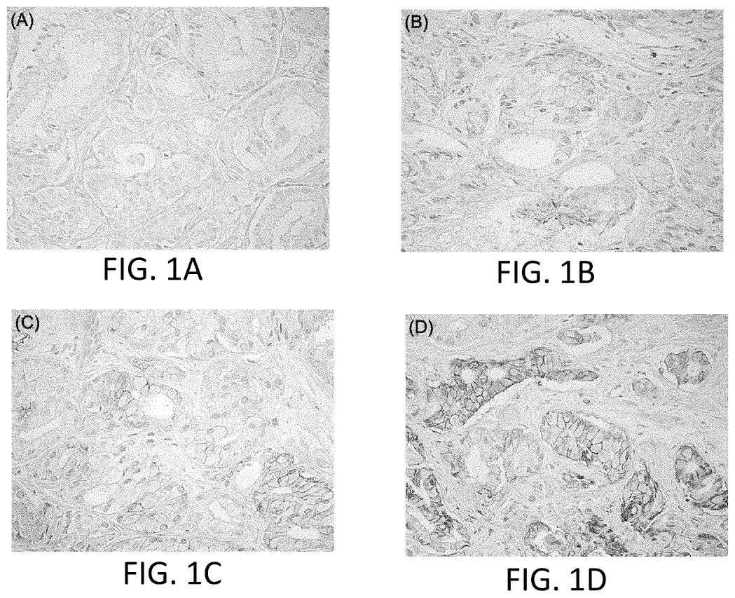

Expression of LAT1, LAT2 and CD98 was assessed as described previously, with minor modifications.[21; 23] The immunointensity of the tumor cell membranes was divided into four categories: 0, no staining; 1, weakly or patchily positive; 2, moderate; and 3, intense complete membrane staining (FIG. 1A-FIG. 1D). Positive staining of the tumor area was classified as: 0, none; 1 (focal), less than 1 mm; 2 (partial), 1-2 mm; and 3 (diffuse), >2 mm. Immunoreactive scores were calculated by multiplying the scores for the area and the highest intensity of positivity. All slides were scored by two pathologists (N.Y. and I.O.) blinded to clinical information, with any disagreements resolved by further review and consensus. Immunoreactive scores of 4 to 9 were classified as high and those of 0 to 3 as low, based on previous results.[20] The number of Ki-67 positive cells per at least 1,000 cells was counted, with Ki-67 LI calculated as a percentage. Ki-67 LIs<3% and ≥3% were classified as low and high, respectively, based on the average Ki-67 LI in all PC lesions (2.9±3.5%) and previous results.[20] The maximum value per patient was used in analyses.

### <Statistical Analysis>

Data were expressed as mean±standard deviation. Groups were compared using Mann-Whitney U test. Correlations among LAT1, LAT2, and CD98 scores and Ki-67 LI were analyzed using Spearman's rank correlation coefficient test, and the relationships between the expression of these proteins and clinicopathological factors were analyzed using Chi-square tests. Logistic regression test was used as a multivariate analysis. StatView software (version 5.0, Abacus Concepts Inc., Berkeley, Calif.) was used for all statistical analyses, with p values <0.05 considered statistically significant.

### <Ethics Approval>

Tissue samples were used with written informed consent of the patients. The study was approved by the Kitasato University School of Medicine and Kitasato University Hospital Ethics Committee (B05-34).

### <Results>

### <Patient Characteristics>

Patient characteristics are shown in Table 3. The mean age at diagnosis of the 109 PC patients was 73.9±6.7 years (range 53-87 years). D'Amico risk classification categorized 19 (18%) as low, 46 (42%) as intermediate and 44 (40%) as high risk. Of the 109 patients, 65 (60%) had stable disease (SD) and 44 (40%) showed LP. These 44 LP patients received deferred definitive or systemic treatment, mainly radiation or hormone therapy, but four (4%) died of the disease. Of the 172 PC lesions, 1 (0.6%) was classified as GS5, 48 (28%) as GS6, 77 (45%) as GS7, 35 (20%) as GS8 and 11 (6%) as GS9, according to the guidelines of the 2005 International Society of Urological Pathology consensus conference.[2]

### <Lat1 Expression>

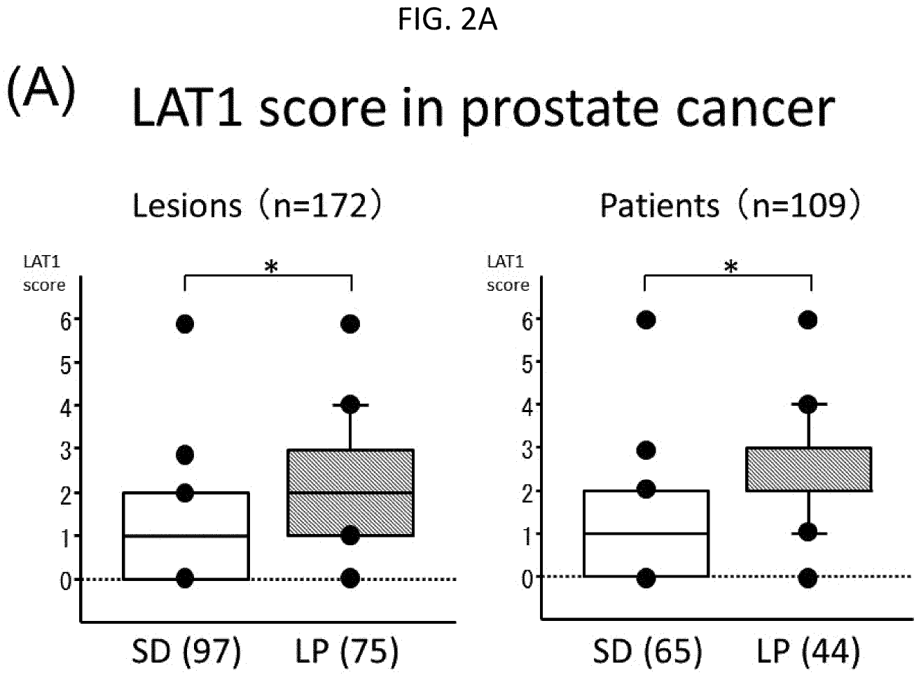

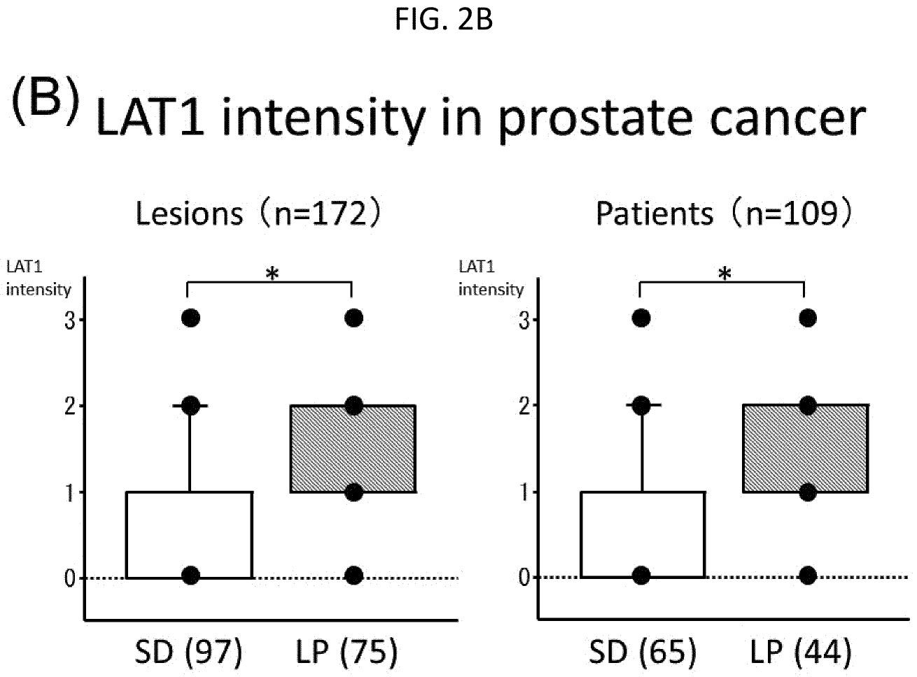

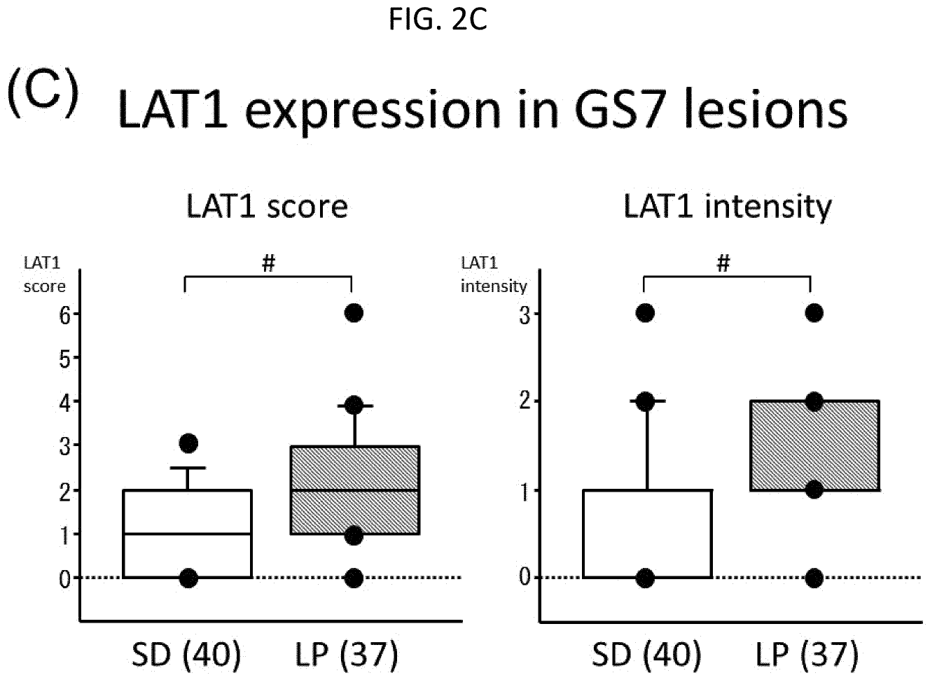

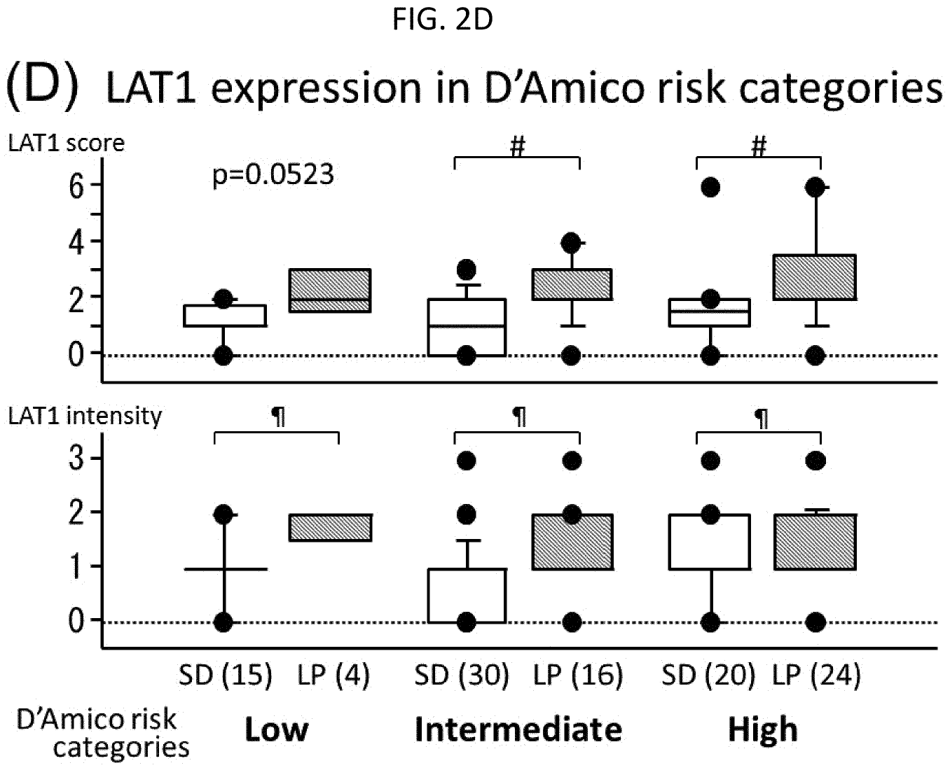

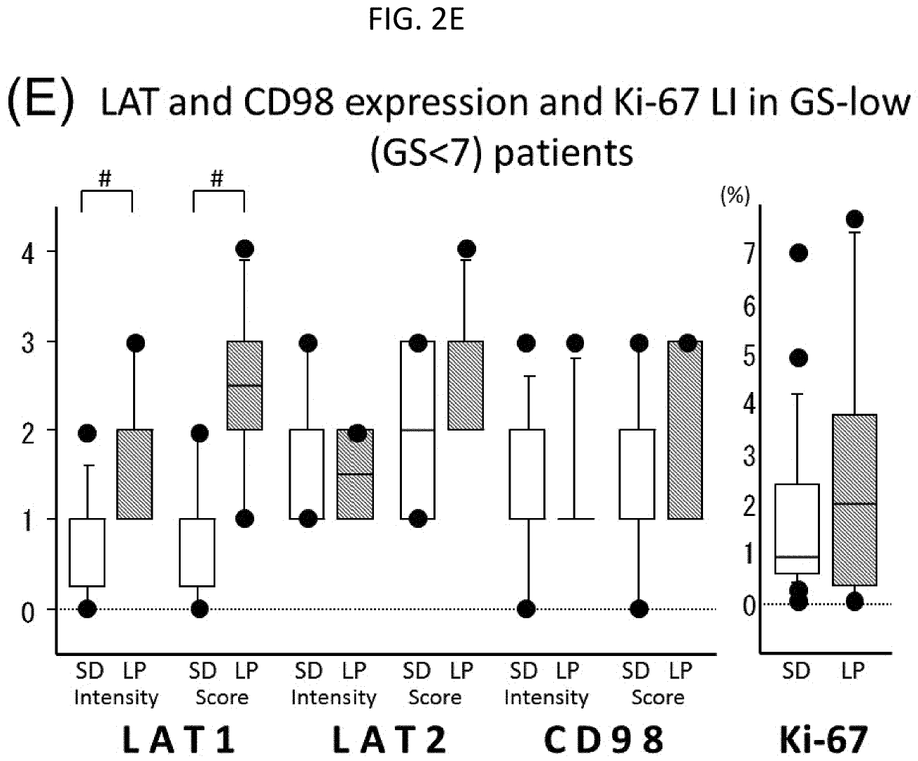

LAT1 expression in normal epithelia of the prostate was none to mild, although some activated lymphocytes showed moderate LAT1 expression. These cells were used as an internal control. Most PC samples showed aberrantly increased LAT1 expression. LP lesions showed significantly higher LAT1 score (2.2±1.4 vs. 1.0±1.0, p<0.0001, FIG. 2A) and intensity (1.4±0.7 vs. 0.8±0.7, p<0.0001, FIG. 2B) than SD lesions. In addition, patients classified as having LP had significantly higher LAT1 score (2.5±1.4 vs. 1.2±1.1; p<0.0001, FIG. 2A) and intensity (1.6±0.7 vs. 0.9±0.7, p<0.0001, FIG. 2B) than patients classified as having SD. Even among GS7 lesions (n=77), those classified as LP had significantly higher LAT1 score (2.2±1.3 vs. 1.1±1.0, p=0.0002) and intensity (1.4±0.7 vs. 0.8±0.7, p=0.0015) than those classified as SD (FIG. 2C). In addition, within each D'Amico risk category, LAT1 scores (low, 2.3±1.3 vs. 1.1±0.7, p=0.0523; intermediate, 2.3±1.1 vs. 1.0±1.0, p=0.0006; high, 2.7±1.6 vs. 1.6±1.3, p=0.0024) and intensities (low, 1.8±0.5 vs. 0.9±0.6, p=0.0241; intermediate, 1.3±0.7 vs. 0.7±0.8, p=0.0114; high, 1.8±0.7 vs. 1.2±0.8, p=0.0113) were significantly higher in patients classified as LP than as SD (FIG. 2D). Finally, among GS-low (GS<7) patients (n=25), those classified as LP (n=6) had significantly higher LAT1 score (2.5±1.0 vs. 0.9±0.7, p=0.0031) and intensity (1.8±0.8 vs. 0.8±0.6, p=0.0072) than those classified as SD (n=19) (FIG. 2E).

### <Expression of LAT2 and CD98 and Ki-67 Labeling Index>

Normal epithelia of the prostate showed no to mild LAT2 membranous expression without any polarity. Similar to LAT1, mild to moderate LAT2 membranous expression was observed in some lymphocytes. LAT2 score (2.8±1.8 vs. 2.1±1.2, p=0.0113) and intensity (1.5±0.6 vs. 1.3±0.6; LP, p=0.0478) were significantly higher in lesions classified as LP than as SD. Moreover, LAT2 score (3.4±2.0 vs. 2.3±1.3, p=0.0026) and intensity (1.6±0.6 vs. 1.4±0.6, p=0.0464) were significantly higher in patients classified as LP than as SD (data not shown). CD98 expression showed the same patterns as LAT1 and LAT2 expression in normal cells and PC, but did not differ between patients or lesions classified as LP and SD (data not shown). Finally, Ki-67 LI was significantly higher in LP than in SD lesions (3.5±4.0% vs. 2.3±3.0%, p=0.0118) and patients (4.4±4.6% vs. 2.6±3.1%, p=0.0063) (data not shown). However, LAT2 and CD98 expression and Ki-67 LI did not differ significantly in GS-low patients categorized as LP or SD (FIG. 2E), as well as in GS7 lesions or in each D'Amico classification group (data not shown).

### <Clinicopathological Features and Immunohistochemical Findings>

The overall results of immunohistochemical analyses are summarized in Table 4. LAT1 and LAT2 expressions, Ki-67 LI, initial PSA and D'Amico risk category differed significantly in patients classified as LP and SD.

TABLE 4
Relation of cliniccpathologic factors to local progression in
patients with prostate cancer under expectant management

| | | | Local | |
|---|---|---|---|---|
| | | Stable | progression | |
| | | cases | cases | |
| | | (n = 65) | (n = 44) | P |
| Age | | | | |
| | <70 y.o. | 17 | 8 | 0.4585 |
| | ≥70 | 48 | 36 | |
| Initial PSA | | | | |
| | Low (<10 ng/ml) | 46 | 17 | 0.0017* |
| | High (≥10) | 19 | 27 | |
| Gleason score | | | | |
| | Low (<7) | 19 | 6 | 0.0949 |
| | High (≥7) | 46 | 38 | |
| Number of | | | | |
| cancer- | | | | |
| containing | ≤3 cores | 51 | 29 | 0.0923 |
| core | | | | |
| | >4 cores | 9 | 13 | |
| LAT1 score | | | | |
| | Low (0-3) | 64 | 35 | 0.0025* |
| | High (4-9) | 1 | 9 | |
| LAT1 intensity | | | | |
| | Low (0-1) | 54 | 19 | <0.0001* |
| | High (2-3) | 11 | 25 | |
| LAT2 score | | | | |
| | Low (0-3) | 57 | 29 | 0.0125* |
| | High (4-9) | 8 | 15 | |
| CD98 score | | | | |
| | Low (0-3) | 58 | 37 | 0.6186 |
| | High (4-9) | 7 | 7 | |
| Ki-67 LI | | | | |
| | Low (<3%) | 50 | 23 | 0.0132* |
| | High (≥3%) | 15 | 21 | |
| D'Amico risk | | | | |
| | Low/Int | 45 | 20 | 0.0223* |
| | High | 20 | 24 | |

Abbreviations: Int, intermediate;
LAT, L-type amino acid transporter;
LI, labeling index;
PSA, prostate-specific antigen.
*p value indicates a significant difference.

### <Correlations Among LAT1, LAT2 and CD98 Expression, Ki-67 LI and Gleason Score>

Immunohistochemically, 16 (9%) lesions showed high intensity of expression of both LAT1 and LAT2, and 15 (9%) showed high intensities of LAT1, LAT2 and CD98. The correlations among LAT1, LAT2 and CD98 expression, Ki-67 LI and GS in PCs are shown in Table 5. LAT2 and CD98 were positively correlated (p=0.525, p<0.0001), as were CD98 and GS (p=0.438, p<0.0001, respectively), whereas correlations between LAT1 and CD98 (p=0.384, p<0.0001) and between LAT2 and GS (p=0.396, p<0.0001) were weaker. No other correlations were found. Especially, LAT1 expression was not correlated with neither GS nor Ki-67 LI.

TABLE 5
Correlations among LAT1, LAT2 and CD98 expression,
Ki-67 Labeling Index and Gleason score

| | ρ | P |
|---|---|---|
| LAT1 score/GS | 0.213 | 0.0053* |
| LAT1 score/LAT2 score | 0.320 | <0.0001* |
| LAT1 score/CD98 score | 0.384 | <0.0001* |
| LAT1 score/Ki-67 LI | 0.208 | 0.0065* |
| LAT2 score/GS | 0.396 | <0.0001* |
| LAT2 score/CD98 score | 0.525 | <0.0001* |
| LAT2 score/Ki-67 LI | 0.160 | 0.368 |
| CD98 score/GS | 0.438 | <0.0001* |
| CD98 score/Ki-67 LI | 0.290 | 0.0002* |
| GS/Ki-67 LI | 0.263 | 0.0006* |

Abbreviations: GS, Gleason score;
LAT, L-type amino acid transporter;
LI, labeling index.
*p value indicates a significant difference.

### <Multivariate Analysis of Correlation Between Clinicopathologic Factors and Local Progression>

In a multivariate logistic regression analysis, LAT1 score had a greater risk for LP (odds ratio, 3.268; 95% confidence interval, 1.794-5.956. Table 6).

TABLE 6
Multivariate logistic regression analysis of
correlation between clinicopathologic factors
and local progression in patients with
prostate cancer under expectant management

| variable | OR (95% CI) | P |
|---|---|---|
| Initial PSA | 1.024 (1.001-1.048) | 0.0378* |
| Gleason score | 1.153 (0.585-2.272) | 0.6803 |
| LAT1 score | 3.268 (1.794-5.956) | 0.0001* |
| LAT2score | 1.504 (1.048-2.159) | 0.0268* |
| CD98 score | 0.569 (0.364-0.889) | 0.0133* |
| Ki-67 LI | 1.107 (0.927-1.321) | 0.2623 |

Abbreviations: CI, confidence interval;
LAT, L-type amino acid transporter;
LI, labeling index;
OR, odds ratio;
PSA, prostate-specific antigen.
*p value indicates a significant difference.

### DISCUSSION

Several recent reports provide convincing evidence that PSA-based PC screening results in considerable overdiagnosis and overtreatment.[31] Serum PSA screening, introduced in the United States after 1986, resulted in the detection of many PCs, even at early stage.[32] However, early detection has been associated with overdiagnosis, since many incidental PCs never progress to cause symptoms or death. Indeed, PSA detection was estimated to avert one death from PC for every 20 men overdiagnosed.[32] In addition, a European trial reported that 1,410 men had to be screened to avoid one PC death.[33] The risks of overdiagnosis and overtreatment may be avoided by strongly distinguishing between aggressive and indolent PCs. The present study found that LAT1 overexpression could predict LP, indicating that LAT1 expression may be a useful biomarker of malignant behavior of PC. Although LAT2 expression and Ki-67 LI may also be prognostic biomarkers, only LAT1 expression differed significantly between LP and SD in GS-low (GS<7) patients, as well as within each D'Amico risk classification group, suggesting that LAT1 may be a superior marker of high grade malignancy. In addition, both high LAT1 score and high LAT1 intensity were associated with LP, suggesting that the presence of high intensity expression of LAT1 by cancer cells is a key factor for tumor progression. Prostate biopsies are usually small samples, limiting the evaluation of tumor area; therefore LAT1 intensity of biopsy samples may be a more reliable prognostic marker of LP. Since this study is retrospective, a prospective trial is also needed.

Elevated serum PSA concentration, including PSADT, has been reported to be a marker of PC growth.[34] PSADT is used as a selection criterion for AS,[31; 35] because preoperative PSA concentration was significantly associated with tumor volume in radical prostatectomy specimens.[36] However, PSA levels alone show low sensitivity and specificity for PC. Although elevated PSA suggests the presence of PC, it also occurs in men with benign conditions of the prostate such as hyperplasia and prostatitis.[37] Further, biopsy-detected PC is not rare among men with PSA concentrations ≤4.0 ng/ml, which are generally thought to be within the normal range.[38] Therefore PSA screening and PSADT assessment alone may miss PC progression. Our findings suggest that immunohistochemical screening of LAT1 expression in prostatic biopsy may be used to identify patients with progressive disease. With the conventional biomarkers such as GS, serum PSA and Ki-67 LI, LAT1 expression might predict LP all together.

LAT1 has been reported expressed in cell membranes of cancer cells of various organs,[17; 18; 19; 20; 21; 23] being thought to actively take up essential amino acids. In contrast, many normal cells ubiquitously express LAT2, the second system L isoform.[16; 39] However, the amino acid specificity and affinity of LAT2 and LAT1 differ.[16] Using a monoclonal antibody against the N-terminal peptide (amino acids 1-52) of LAT1, we found that high-LAT1 expression was associated with progressive PC, similar to findings in other cancers.[18; 19; 20; 21; 23] Moreover, several LAT1 inhibitors have been reported to inhibit the growth of cancer cell lines. One of these inhibitors, JPH203 (KYT-0353), significantly inhibited the growth of human colon cancer cells both in vitro and in vivo,[40] and another inhibitor, 2-aminobicyclo-(2,2,1)-heptane-2-carboxylic acid, reduced the viability of lung cancer cells,[41] suggesting that LAT1 inhibitors may be clinically useful in cancer chemotherapy. These results indicate that LAT1 inhibitors are effective, especially against human malignancies that express high levels of LAT1.

We previously demonstrated that LAT1 expression could be a reliable prognostic marker in PC.[20] Other groups reported a significant correlation between LAT1 expression and GS.[42] However, both our previous and current studies found no significant correlation between LAT1 expression and GS.[20] These discrepancies may be due to differences in samples assayed, or the use of biopsy or radical prostatectomy specimens. Although it is reasonable that tumor cells with high proliferative activity showed LAT1 overexpression, GS is a system of histological grading based on the overall growth pattern of the tumor examined at low magnification.[4] Therefore, GS is thought to be more strongly associated with tumor differentiation than proliferative activity, differently from LAT1 expression. In agreement with our findings, no association has been observed between LAT1 expression and tumor differentiation in gastric, pancreatic and bile duct cancers.[18; 21; 23] Although not observed in this study, LAT1 expression has been found to correlate significantly with Ki-67 LI,[18; 19; 43] suggesting a closer association between LAT1 and proliferative activity. LAT1 expression and GS may complement each other for the evaluation of PC to predict LP.

LAT expression has been reported in human PC cell lines. Moreover, increased LAT3 expression has been observed in primary PC and increased LAT1 expression in metastases.[44] Androgen receptor signaling may activate LAT3 transcription in primary PC, whereas decreased androgen signaling and LAT3 expression resulting from hormone ablation therapy leading to ATF4 translation, may initiate LAT1 transcription.[44] Knockdown of either LAT3 or LAT1 expression in PC cell lines has been found to inhibit mTORC1 pathway activation, as well as cell growth and the cell cycle both in vitro and in vivo[45], indicating the importance of LAT in PC cells. Interestingly, we observed aberrant LAT2 expression immunohistochemically in PC for the first time. We could not investigate LAT3 in human PC tissue, suggesting the need for additional studies.

### CONCLUSION

In conclusion, our findings suggested that elevated LAT1 expression in PC is a novel biomarker for high-grade malignancy. Independently of GS, aberrant LAT1 overexpression might be used to screen for aggressive phenotypes of PC that should be treated medically. Prostate biopsies are usually small samples, limiting the evaluation of the tumor area. Thus, LAT1 intensity in prostate biopsy samples may be more a reliable prognostic marker of LP. Especially, we propose LAT1 evaluation against PC with low-risk patients in order to screen who can receive active surveillance. Several LAT1 inhibitors have been found to suppress cancer cell proliferation, so inhibition of LAT1 may be a potential therapeutic strategy for PC and other human cancers.

### REFERENCES

[1] R. Siegel, J. Ma, Z. Zou, and A. Jemal, Cancer statistics, 2014. CA Cancer J Clin 64 (2014), 9-29.
[2] J. I. Epstein, W. C. Allsbrook, Jr., M. B. Amin, and L. L. Egevad, The 2005 International Society of Urological Pathology (ISUP) Consensus Conference on Gleason Grading of Prostatic Carcinoma. Am J Surg Pathol 29 (2005), 1228-1242.
[3] D. F. Gleason, Histologic grading of prostate cancer: a perspective. Hum Pathol 23 (1992), 273-279.
[4] D. F. Gleason, and G. T. Mellinger, Prediction of prognosis for prostatic adenocarcinoma by combined histological grading and clinical staging. J Urol 111 (1974), 58-64.
[5] D. G. Bostwick, Gleason grading of prostatic needle biopsies. Correlation with grade in 316 matched prostatectomies. Am J Surg Pathol 18 (1994), 796-803.
[6] S. E. Spires, M. L. Cibull, D. P. Wood, Jr., S. Miller, S. M. Spires, and E. R. Banks, Gleason histologic grading in prostatic carcinoma. Correlation of 18-gauge core biopsy with prostatectomy. Arch Pathol Lab Med 118 (1994), 705-708.
[7] J. C. Presti, Jr., K. Shinohara, P. Bacchetti, V. Tigrani, and V. Bhargava, Positive fraction of systematic biopsies predicts risk of relapse after radical prostatectomy. Urology 52 (1998), 1079-1084.
[8] R. A. Badalament, M. C. Miller, P. A. Peller, D. C. Young, D. K. Bahn, P. Kochie, G. J. O'Dowd, and R. W. Veltri, An algorithm for predicting nonorgan confined prostate cancer using the results obtained from sextant core biopsies with prostate specific antigen level. J Urol 156 (1996), 1375-1380.
[9] M. K. Terris, D. J. Haney, I. M. Johnstone, J. E. McNeal, and T. A. Stamey, Prediction of prostate cancer volume using prostate-specific antigen levels, transrectal ultrasound, and systematic sextant biopsies. Urology 45 (1995), 75-80.
[10] A. de la Taille, M. A. Rubin, E. Bagiella, C. A. Olsson, R. Buttyan, T. Burchardt, C. Knight, K. M. O'Toole, and A. E. Katz, Can perineural invasion on prostate needle biopsy predict prostate specific antigen recurrence after radical prostatectomy? J Urol 162 (1999), 103-106.
[11] N. Yanagisawa, R. Li, D. Rowley, H. Liu, D. Kadmon, B. J. Miles, T. M. Wheeler, and G. E. Ayala, Stromogenic prostatic carcinoma pattern (carcinomas with reactive stromal grade 3) in needle biopsies predicts biochemical recurrence-free survival in patients after radical prostatectomy. Hum Pathol 38 (2007), 1611-1620.
[12] R. Arora, M. O. Koch, J. N. Eble, T. M. Ulbright, L. Li, and L. Cheng, Heterogeneity of Gleason grade in multifocal adenocarcinoma of the prostate. Cancer 100 (2004), 2362-2366.
[13] P. D. Sved, P. Gomez, M. Manoharan, S. S. Kim, and M. S. Soloway, Limitations of biopsy Gleason grade: implications for counseling patients with biopsy Gleason score 6 prostate cancer. J Urol 172 (2004), 98-102.
[14] A. Heidenreich, P. J. Bastian, J. Bellmunt, M. Bolla, S. Joniau, T. van der Kwast, M. Mason, V. Matveev, T. Wiegel, F. Zattoni, and N. Mottet, EAU guidelines on prostate cancer. part 1: screening, diagnosis, and local treatment with curative intent-update 2013. Eur Urol 65 (2014), 124-137.
[15] A. E. Ross, S. Loeb, P. Landis, A. W. Partin, J. I. Epstein, A. Kettermann, Z. Feng, H. B. Carter, and P. C. Walsh, Prostate-specific antigen kinetics during follow-up are an unreliable trigger for intervention in a prostate cancer surveillance program. J Clin Oncol 28 (2010), 2810-2816.
[16] H. Segawa, Y. Fukasawa, K. Miyamoto, E. Takeda, H. Endou, and Y. Kanai, Identification and functional characterization of a Na+-independent neutral amino acid transporter with broad substrate selectivity. J Biol Chem 274 (1999), 19745-19751.
[17] Y. Kanai, H. Segawa, K. Miyamoto, H. Uchino, E. Takeda, and H. Endou, Expression cloning and characterization of a transporter for large neutral amino acids activated by the heavy chain of 4F2 antigen (CD98). J Biol Chem 273 (1998), 23629-23632.
[18] M. Ichinoe, T. Mikami, T. Yoshida, I. Igawa, T. Tsuruta, N. Nakada, N. Anzai, Y. Suzuki, H. Endou, and I. Okayasu, High expression of L-type amino-acid transporter 1 (LAT1) in gastric carcinomas: comparison with non-cancerous lesions. Pathol Int 61 (2011), 281-289.
[19] K. Kaira, N. Oriuchi, H. Imai, K. Shimizu, N. Yanagitani, N. Sunaga, T. Hisada, T. Ishizuka, Y. Kanai, T. Nakajima, and M. Mori, Prognostic significance of L-type amino acid transporter 1 (LAT1) and 4F2 heavy chain (CD98) expression in stage I pulmonary adenocarcinoma. Lung Cancer 66 (2009), 120-126.
[20] T. Sakata, G. Ferdous, T. Tsuruta, T. Satoh, S. Baba, T. Muto, A. Ueno, Y. Kanai, H. Endou, and I. Okayasu, L-type amino-acid transporter 1 as a novel biomarker for high-grade malignancy in prostate cancer. Pathol Int 59 (2009), 7-18.
[21] N. Yanagisawa, M. Ichinoe, T. Mikami, N. Nakada, K. Hana, W. Koizumi, H. Endou, and I. Okayasu, High expression of L-type amino acid transporter 1 (LAT1) predicts poor prognosis in pancreatic ductal adenocarcinomas. J Clin Pathol 65 (2012), 1019-1023.
[22] N. Nakada, T. Mikami, K. Hana, M. Ichinoe, N. Yanagisawa, T. Yoshida, H. Endou, and I. Okayasu, Unique and selective expression of L-amino acid transporter 1 in human tissue as well as being an aspect of oncofetal protein. Histol Histopathol 29 (2014), 217-227.
[23] N. Yanagisawa, K. Hana, N. Nakada, M. Ichinoe, W. Koizumi, H. Endou, I. Okayasu, and Y. Murakumo, High expression of L-type amino acid transporter 1 as a prognostic marker in bile duct adenocarcinomas. Cancer Med 3 (2014), 1246-1255.
[24] M. F. Wempe, P. J. Rice, J. W. Lightner, P. Jutabha, M. Hayashi, N. Anzai, S. Wakui, H. Kusuhara, Y. Sugiyama, and H. Endou, Metabolism and pharmacokinetic studies of JPH203, an L-amino acid transporter 1 (LAT1) selective compound. Drug Metab Pharmacokinet 27 (2014), 155-161.
[25] D. W. Yun, S. A. Lee, M. G. Park, J. S. Kim, S. K. Yu, M. R. Park, S. G. Kim, J. S. Oh, C. S. Kim, H. J. Kim, H. S. Chun, Y. Kanai, H. Endou, M. F. Wempe, and K. Kim do, JPH203, an L-type amino acid transporter 1-selective compound, induces apoptosis of YD-38 human oral cancer cells. J Pharmacol Sci 124 (2014), 208-217.
[26] Y. Asano, Y. Inoue, Y. Ikeda, K. Kikuchi, T. Hara, C. Taguchi, T. Tokushige, H. Maruo, T. Takeda, T. Nakamura, T. Fujita, Y. Kumagai, and K. Hayakawa, Phase I clinical study of NMK36: a new PET tracer with the synthetic amino acid analogue anti-[18F]FACBC. Ann Nucl Med 25 (2011), 414-418.
[27] Y. Inoue, Asano. Y., Satoh, T., Tabata, K., Kikuchi, K., Woodhams, R., Baba, S., Hayakawa, K., Phase IIa Clinical Trial of Trans-1-Amino-3-18F-Fluoro-Cyclobutane Carboxylic Acid in Metastatic Prostate Cancer. Asia Oceania J Nucl Med Biol 2 (2014), 87-94.
[28] L. Sobin, M. K. Gospodarowicz, and C. Wittekind, (Eds.), UICC; TNM Classification of Malignant Tumors, 7th edn., Wiley-Blackwell, Oxford, 2009.
[29] R. T. Vollmer, S. Egawa, S. Kuwao, and S. Baba, The dynamics of prostate specific antigen during watchful waiting of prostate carcinoma: a study of 94 Japanese men. Cancer 94 (2002), 1692-1698.
[30] R. Kurayama, N. Ito, Y. Nishibori, D. Fukuhara, Y. Akimoto, E. Higashihara, Y. Ishigaki, Y. Sai, K. Miyamoto, H. Endou, Y. Kanai, and K. Yan, Role of amino acid transporter LAT2 in the activation of mTORC1 pathway and the pathogenesis of crescentic glomerulonephritis. Lab Invest 91 (2011), 992-1006.
[31] L. H. Klotz, Active surveillance with selective delayed intervention: walking the line between overtreatment for indolent disease and undertreatment for aggressive disease. Can J Urol 12 Suppl 1 (2005), 53-57; discussion 101-102.
[32] H. G. Welch, and P. C. Albertsen, Prostate cancer diagnosis and treatment after the introduction of prostate-specific antigen screening: 1986-2005. J Natl Cancer Inst 101 (2009), 1325-1329.
[33] G. Draisma, R. Boer, S. J. Otto, I. W. van der Cruijsen, R. A. Damhuis, F. H. Schroder, and H. J. de Koning, Lead times and overdetection due to prostate-specific antigen screening: estimates from the European Randomized Study of Screening for Prostate Cancer. J Natl Cancer Inst 95 (2003), 868-878.
[34] H. P. Schmid, J. E. McNeal, and T. A. Stamey, Observations on the doubling time of prostate cancer. The use of serial prostate-specific antigen in patients with untreated disease as a measure of increasing cancer volume. Cancer 71 (1993), 2031-2040.
[35] Y. Kakehi, T. Kamoto, T. Shiraishi, O. Ogawa, Y. Suzukamo, S. Fukuhara, Y. Saito, K. Tobisu, T. Kakizoe, T. Shibata, H. Fukuda, K. Akakura, H. Suzuki, N. Shinohara, S. Egawa, A. Irie, T. Sato, O. Maeda, N. Meguro, Y. Sumiyoshi, T. Suzuki, N. Shimizu, Y. Arai, A. Terai, T. Kato, T. Habuchi, H. Fujimoto, and M. Niwakawa, Prospective evaluation of selection criteria for active surveillance in Japanese patients with stage T1cNOMO prostate cancer. Jpn J Clin Oncol 38 (2008), 122-128.
[36] T. A. Stamey, and J. N. Kabalin, Prostate specific antigen in the diagnosis and treatment of adenocarcinoma of the prostate. I. Untreated patients. J Urol 141 (1989), 1070-1075.
[37] W. J. Catalona, D. S. Smith, T. L. Ratliff, K. M. Dodds, D. E. Coplen, J. J. Yuan, J. A. Petros, and G. L. Andriole, Measurement of prostate-specific antigen in serum as a screening test for prostate cancer. N Engl J Med 324 (1991), 1156-1161.
[38] I. M. Thompson, D. K. Pauler, P. J. Goodman, C. M. Tangen, M. S. Lucia, H. L. Parnes, L. M. Minasian, L. G. Ford, S. M. Lippman, E. D. Crawford, J. J. Crowley, and C. A. Coltman, Jr., Prevalence of prostate cancer among men with a prostate-specific antigen level < or =4.0 ng per milliliter. N Engl J Med 350 (2004), 2239-2246.
[39] M. Pineda, E. Fernandez, D. Torrents, R. Estevez, C. Lopez, M. Camps, J. Lloberas, A. Zorzano, and M. Palacin, Identification of a membrane protein, LAT-2, that Co-expresses with 4F2 heavy chain, an L-type amino acid transport activity with broad specificity for small and large zwitterionic amino acids. J Biol Chem 274 (1999), 19738-19744.
[40] K. Oda, N. Hosoda, H. Endo, K. Saito, K. Tsujihara, M. Yamamura, T. Sakata, N. Anzai, M. F. Wempe, Y. Kanai, and H. Endou, L-type amino acid transporter 1 inhibitors inhibit tumor cell growth. Cancer Sci 101 (2010), 173-179.
[41] H. Imai, K. Kaira, N. Oriuchi, K. Shimizu, H. Tominaga, N. Yanagitani, N. Sunaga, T. Ishizuka, S. Nagamori, K. Promchan, T. Nakajima, N. Yamamoto, M. Mori, and Y. Kanai, Inhibition of L-type amino acid transporter 1 has antitumor activity in non-small cell lung cancer. Anticancer Res 30 (2010), 4819-4828.
[42] A. Segawa, S. Nagamori, Y. Kanai, N. Masawa, and T. Oyama, L-type amino acid transporter 1 expression is highly correlated with Gleason score in prostate cancer. Mol Clin Oncol 1 (2013), 274-280.
[43] H. Imai, K. Kaira, N. Oriuchi, N. Yanagitani, N. Sunaga, T. Ishizuka, Y. Kanai, H. Endou, T. Nakajima, and M. Mori, L-type amino acid transporter 1 expression is a prognostic marker in patients with surgically resected stage I non-small cell lung cancer. Histopathology 54 (2009), 804-813.
[44] Q. Wang, C. G. Bailey, C. Ng, J. Tiffen, A. Thoeng, V. Minhas, M. L. Lehman, S. C. Hendy, G. Buchanan, C. C. Nelson, J. E. Rasko, and J. Holst, Androgen receptor and nutrient signaling pathways coordinate the demand for increased amino acid transport during prostate cancer progression. Cancer Res 71 (2011), 7525-7536.
[45] Q. Wang, T. Grkovic, J. Font, S. Bonham, R. H. Pouwer, C. G. Bailey, A. M. Moran, R. M. Ryan, J. E. Rasko, M. Jormakka, R. J. Quinn, and J. Holst, Monoterpene Glycoside ESK246 from Pittosporum Targets LATS Amino Acid Transport and Prostate Cancer Cell Growth. ACS Chem Biol 9 (2014), 1369-1376.

1. A method for determining prostate cancer malignancy, comprising:
applying an anti-human LAT1 monoclonal antibody to a specimen tissue of prostate cancer from a patient classified as Gleason score <7 or Low according to D'Amico Risk Category;
performing immunohistochemical staining on the specimen tissue to determine the LAT1 score and/or intensity; and
determining whether the prostate cancer of the patient is in stable disease or local progression based on the score and/or intensity.

2. The method for determining prostate cancer malignancy according to claim 1, wherein the monoclonal antibody recognizes human LAT1 amino acid residues specifically at positions 1 to 52 from the N-terminus.

3. A method to clinically differentiate prostate cancer severity via application of LAT1 molecular target therapeutic agent(s), comprising:
determining malignancy of prostate cancer according to the method as claimed in claim 1; and
determining whether a therapeutic agent for prostate cancer is to be administered or not, based on the determination result.

4. A method to clinically differentiate prostate cancer severity via application of LAT1 molecular target therapeutic agent(s), comprising:
determining malignancy of prostate cancer according to the method as claimed in claim 2; and
determining whether a therapeutic agent for prostate cancer is to be administered or not, based on the determination result.
